(19) United States
(12) Patent Application Publication
(10) Pub. No.: US 2019/0125099 A1
(43) Pub. Date: May 2, 2019

(54) ANTI-SIDS INFANT CRIB

(71) Applicant: Madoc EL MALI, Rishon Lezion (IL)

(72) Inventor: Madoc EL MALI, Rishon Lezion (IL)

(21) Appl. No.: 16/172,901

(22) Filed: Oct. 29, 2018

(30) Foreign Application Priority Data
Oct. 30, 2017 (IL) 255340

(51) Int. Cl. A47D 9/00; A47G 9/10; A61B 5/00; A61B 5/113

(52) U.S. Cl. CPC A47D 9/00; A47G 9/1009; A47D 15/001; A61B 5/113; A61B 5/6891

(57) ABSTRACT

An anti-SIDS infant crib, comprising: a crib (10); a processor (20); one or more SIDS-related sensors (38), installed at the crib's side, and being in communication with the processor (20); an elastic and adjustable belt (30) surrounding an infant in the crib, for preventing the infant from turning upside-down while allowing a limited ability to the infant to turn over; wherein the belt hosting one or more sensors that come in contact with an infant body or leotard; wherein the processor being adapted to identify an SIDS alerting state by a combination and threshold of the sensors (38); a remote control (50), in wireless communication (60) with the processor (20), the remote control being adapted for monitoring an operation of the processor (20), and alerting a user upon identifying an alerting state by the processor; thereby providing to a parent SIDS-control and alerts of an infant lying in the crib.

### TECHNICAL FIELD

The present invention relates to the field of SIDS prevention.

### BACKGROUND ART

“Sudden infant death syndrome (SIDS), also known as cot death or crib death, is the sudden unexplained death of a child less than one year of age. —SIDS usually occurs during sleep.—Placing an infant to sleep while lying on the stomach or the side increases the risk. This increased risk is greatest at two to three months of age.” (From Wikipedia)

U.S. Pat. No. 8,333,560 discloses a portable fan that can attach to a baby crib in order to provide air movement across an infant in order to prevent the likelihood of sudden infant death syndrome.

U.S. 5,389,037 discloses a method and apparatus for reducing respiratory abnormalities in infants and the incidence of crib death by providing a flow of room air to a sleeping infant's environment to stimulate breathing. In a preferred embodiment of the invention, room air is delivered into a crib through an air plenum that is removably attached to the vertical bars of a crib and positioned within 1 cm to 20 cm of the infant's mouth, nose, larynx and trachea. The flow of room air from the air plenum safely assists the respiration of an infant with inadequate shallow end tidal volume, decreases rebreathing of expired carbon dioxide from the nose, mouth, oral cavity and trachea, prevents pockets of increasing carbon dioxide from developing within the cushions and beddings surrounding the infant, and decreases the likelihood of overheating and other conditions associated with sudden infant death syndrome, apnea syndromes and hypoventilation.

U.S. 5,483,711 discloses a safety pad or mattress such as for use in a crib prevents sudden infant death syndrome by ensuring an oxygenated breathing space beneath the infant. Reticulated foam or other air permeable material is made into the shape of a pad or even a mattress and covered with a fitted open weave fabric covering. An air tube is embedded in the pad or mattress and interconnected with an air pump which circulates fresh, i.e., oxygenated, air in a breathing space formed beneath an infant by the air permeable mattress. The forced air circulation flushes any exhaled carbon dioxide from the breathing space, even when the infant is face down or otherwise in a prone position on the mattress, to prevent carbon dioxide poisoning.

All the methods described above have not yet provided satisfactory solutions to the problem of preventing crib death.

It is an object of the present invention to provide a solution to the above-mentioned and other problems of the prior art.

Other objects and advantages of the invention will become apparent as the description proceeds.

### SUMMARY OF THE INVENTION

The present invention is directed to an anti-SIDS infant crib, comprising:
a crib (10);
a processor (20);
one or more SIDS-related sensors (38), installed at the crib's side, and being in communication with the processor (20);
an elastic and adjustable belt (30) surrounding an infant in the crib, for preventing the infant from turning upside-down while allowing a limited ability to the infant to turn over;
wherein the belt hosting one or more sensors that come in contact with an infant body or leotard;
wherein the processor being adapted to identify an SIDS alerting state by a combination and threshold of the sensors (38);
a remote control (50), in wireless communication (60) with the processor (20), the remote control being adapted for monitoring an operation of the processor (20), and alerting a user upon identifying an alerting state by the processor;
thereby providing to a parent SIDS-control and alerts of an infant lying in the crib.

According to one embodiment of the invention, the sensors (38) include (a) a camera (16) and microphone (62), for taking images of an infant lying in the crib, and displaying thereof on a remote display, and (b) warnings.

The anti-SIDS infant crib may further comprise a multimedia system (42), disposed at the crib's side, for displaying content to the infant.

The anti-SIDS infant crib may further comprise a screen (12), for displaying to the infant video presentations.

The anti-SIDS infant crib may further comprise an adjustable mattress (14), for allowing adapting thereof so that an infant lying in the crib can view the screen.

The anti-SIDS infant crib may further comprise a baby bottle heater facility (22) controllable by the processor (20), for heating a baby bottle (36) disposed therein.

The one or more sensors (38) that come in contact with an infant body or leotard may be a heartbeat rate sensor, a breath rate sensor, oxygen in blood sensor, hygrometer, a thermometer for metering a temperature of the infant, a thermometer for metering a temperature of the room.

According to one embodiment of the invention, the one or more sensors (38) include a microphone. In this case the processor may be further adapted to detect an infant's cry.

One of the sensors that can be added is a length meter in a form of a track (28) disposed at the inner side of the crib's walls, and a length counter (26) slideable along the track, thereby allowing measuring a height of the infant when lying in the crib.

According to one embodiment of the invention, the one or more sensors (38) include a weight meter, for metering a weight of the infant.

The anti-SIDS infant crib may further comprise means for storing results of measurements, thereby allowing diagnosing abnormal development of an infant lying in the crib.

The anti-SIDS infant crib may further comprise means for transmitting measurements, results to a remote database, and updating said processor of thresholds and combinations of alerting states.

Preferably, the belt further comprises a vibration mechanism for vibrating said belt upon identifying said SIDS alerting state.

According to one embodiment of the invention, the belt further comprises a display (34), for displaying measurements, states, and instructions, and the like.

### BRIEF DESCRIPTION OF DRAWINGS

Preferred embodiments, features, aspects and advantages of the present invention are described herein in conjunction with the following drawings:

FIG. 1 is a perspective view which pictorially illustrates an anti-SIDS infant crib, according to one embodiment of the invention.

FIG. 2 is a front view thereof.

FIG. 3a is a longitudinal sectional view of an anti-SIDS infant crib, according to one embodiment of the invention.

FIG. 3b is a longitudinal sectional view of an anti-SIDS infant crib, such that the left side of the mattress (in the illustration's orientation) is inclined, according to one embodiment of the invention.

FIG. 4 pictorially illustrates an infant lying in an anti-SIDS infant crib, according to one embodiment of the invention.

FIG. 5 pictorially illustrates a remote control of an anti-SIDS infant crib, according to one embodiment of the invention.

FIG. 6 is a block diagram illustrating the data flow in an anti-SIDS infant crib, according to one embodiment of the invention.

FIG. 7a is a latitudinal cross-section of an anti-SIDS infant crib in which the infant lies on his back, according to one embodiment of the invention.

FIG. 7b is a latitudinal cross-section of an anti-SIDS infant crib in which the infant tries to turn upside-down while the elastic belt prevents it, according to the same embodiment of the invention.

It should be understood that the drawings are not necessarily drawn to scale.

### DESCRIPTION OF EMBODIMENTS

The present invention will be understood from the following detailed description of preferred embodiments (“best mode”), which are meant to be descriptive and not limiting. For the sake of brevity, some well-known features, methods, systems, procedures, components, circuits, and so on, are not described in detail.

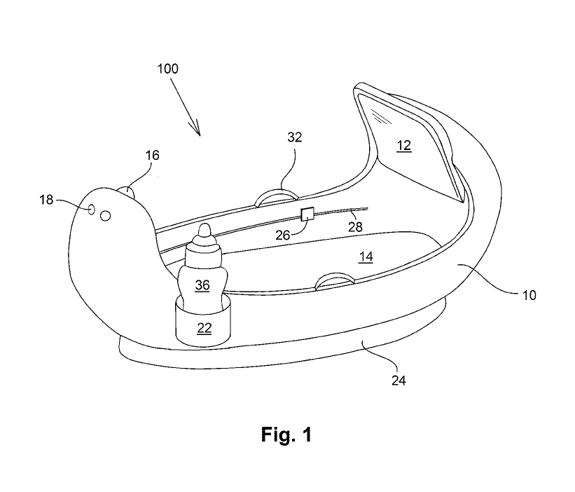

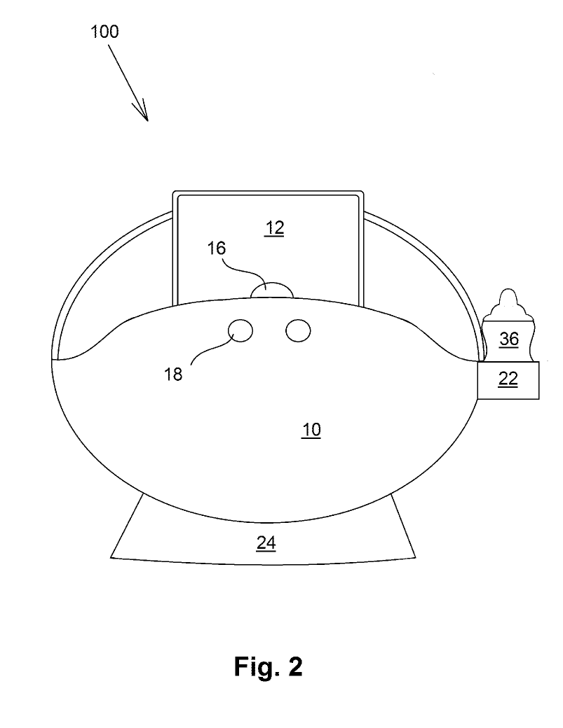

FIG. 1 is a perspective view which pictorially illustrates an anti-SIDS infant crib, according to one embodiment of the invention. FIG. 2 is a front view thereof.

Preferably, the contour of the crib 10 is of an egg, which people, especially adults, associate with primacy.

The crib comprises carrying handles 32, by which a parent can move the crib and place it in another location.

A display is disposed against the face of the infant lying in the crib, for displaying thereon graphical presentations, films, and the like, which may interest the infant. The display may be a computer monitor, a tablet computer, a television display, and the like.

The crib comprises a mattress 14 which is composed of two or more parts, so that the angle between them can be changed. In this way, the position of the infant lying on the mattress can be adjusted to allow him a better view of the screen 12.

The angle may be changed manually or by electromechanical means (not illustrated).

The angle change may be activated remotely or by local input means such as by button, knobs, and so on (not illustrated).

Preferably, in the crib is installed a multimedia system comprising at least audio display means and speakers, which preferably are disposed at the sides of the infant's head. The multimedia system may use a display, such as screen 12.

Above the head of the infant is disposed a camera 16, through which a parent can watch the infant at a distance. Preferably, the camera comprises night vision capability, in order to allow the parent to clearly see the infant at night time.

A baby bottle heater facility 22 is installed on a wall of the crib, whether inside or outside the crib. A parent can put the baby food bottle inside the facility, and at feeding time the bottle will be warm. Preferably, the bottle heater comprises means for adjusting the required heating temperature.

The crib comprises a length meter, for measuring the infant's length when lying (i.e., its height). As will be detailed hereinafter, the length meter may be embodied as a length counter 26 (trailer) slideable along a track 28 at the inner side of the crib's walls. The length counter (trailer) may be as a longitudinal digital micrometer. Thus, the parent places the trailer near the infant top, sets the counter to zero, and drags the trailer to the bottom of the infant. While dragging the trailer, the distance is displayed on its digital display. In order to allow a parent to point on the top and bottom points of the infant, the counter 26 may further comprise a vertical bar (not illustrated).

A user may press a “SAVE” button in order to store the recent metered measurement and its date in a database.

At the outer side of the crib is installed one or more lamps 18 for providing background light around the crib. The color and intensity of the lighting of the lamps may be controlled by appropriate input means (knobs and the like).

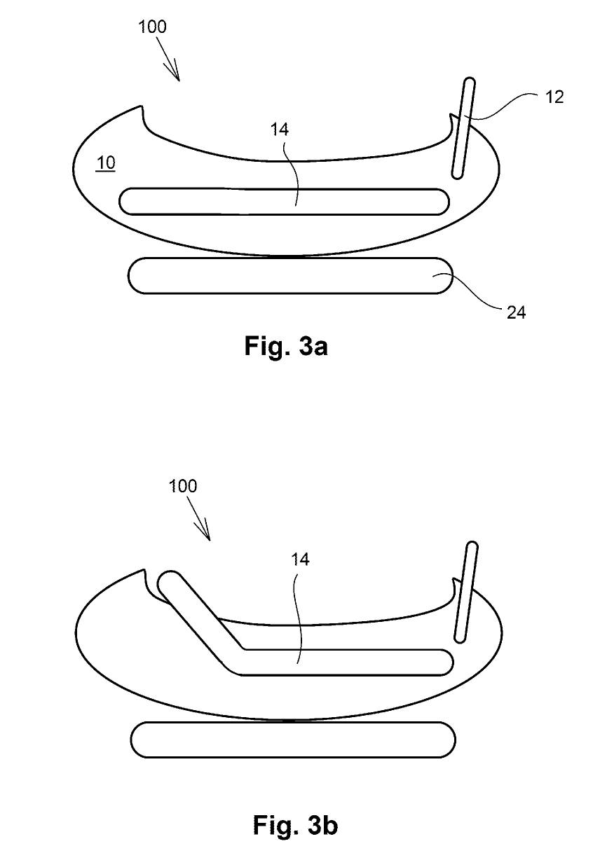

FIG. 3a is a longitudinal sectional view of an anti-SIDS infant crib, according to one embodiment of the invention.

As mentioned, the crib comprises a mattress 14 which is composed of two or more parts, so that the angle between them can be changed. In this way, the position of the infant lying on the mattress can be adjusted to allow him a better view of the screen 12.

FIG. 3b is a longitudinal sectional view of an anti-SIDS infant crib, such that the left side of the mattress (in the illustration's orientation) is inclined, according to one embodiment of the invention.

The inclination angle may be changed manually or by electromechanical means (not illustrated).

The inclination angle may be controlled remotely or by local input means such as by button, knobs, and so on (not illustrated).

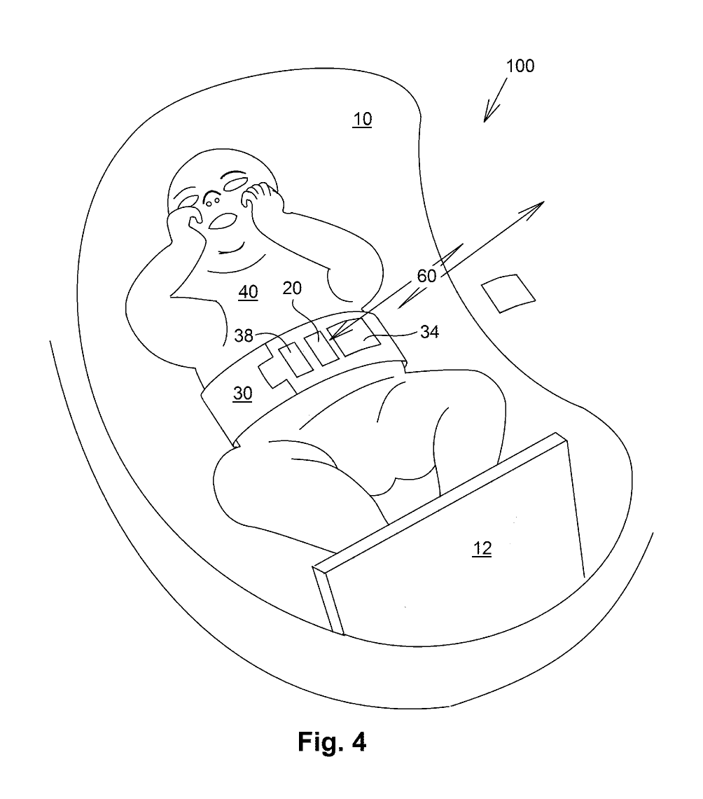

FIG. 4 pictorially illustrates an infant lying in an anti-SIDS infant crib, according to one embodiment of the invention.

The infant 40 wears a belt 30 on which may be installed a plurality of sensors 38, such as heartbeat rate sensor, breath rate sensor, hygrometer, and actually any sensor that comes with contact with an infant body or leotard.

As per the breath rate sensor, since the belt is tensed, and the tension changes along with the infant's breath, these changes may be used for metering the breath rate.

Additional sensors that can be installed on the belt, but also can be installed elsewhere in the crib, are a thermometer, a microphone, a weight meter, and the like.

The sensors are connected to a processor 20 which communicates with a remote control (not shown) via wireless communication 60, with a smart phone and the like (not shown). Preferably, the remote control is also used as an alerting device. Alternatively, the alerting device may be embedded in the processor.

When one of the sensors senses an exceptional measurement (e.g., the metered value is compared to a predefined threshold, or a certain combination of the sensing of several sensors), the system alerts the parent via the alerting device.

As mentioned, the heartbeat and breath rate sensors 38 may be embedded in the belt. Actually, any sensor which comes in contact with the infant body or leotard is preferably installed in the belt. The hygrometer may also be disposed in the diaper of the infant, and connected to the processor 20 by wired or unwired communication means.

The weight sensor may be disposed under the mattress, or on the mattress. The same regards the microphone. The sound pattern sampled by the microphone may be compared to a pattern of baby cries, and when the infant cries, the parent may be alerted. The alert may include an indication of the intensity of the cry. For example, the density beep sounds may indicate the strength of the cry, and so on.

One of the major features of the invention is the ability to prevent an infant lying in the crib from rolling on his side or stomach. Such means may be the elastic belt 30. As mentioned, keeping the infant lying on his back decreases the chance of the infant dying from crib death.

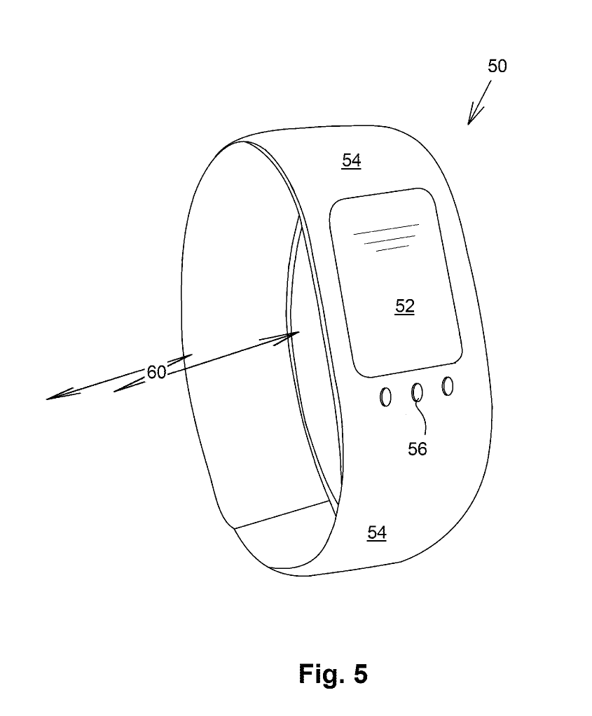

FIG. 5 pictorially illustrates a remote control of an anti-SIDS infant crib, according to one embodiment of the invention.

The remote control 50 comprises wrist belt 54, a display 52, and operational buttons 56. The wrist belt 54 allows a user to wear the remote control 50 on his wrist.

The remote control 50 also comprises a processor (not shown) which communicates with the processor 20 (not shown) on the crib via wireless communication 60.

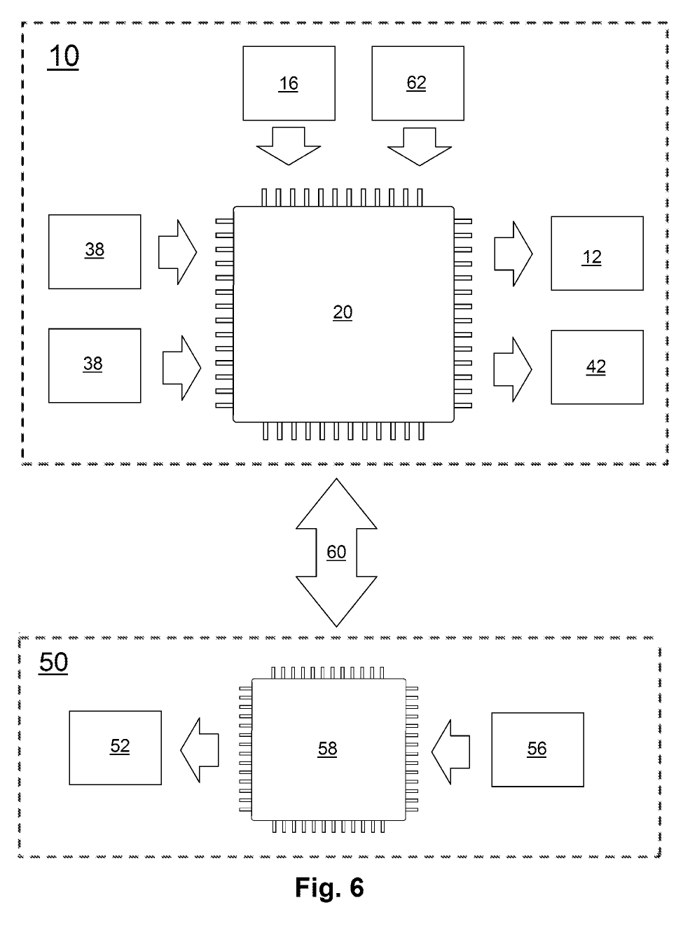

FIG. 6 is a block diagram illustrating the data flow in an anti-SIDS infant crib, according to one embodiment of the invention.

At the crib site is installed a processor 20 which gets input from sensors 38, camera 16, microphone 62, and the like.

The sensors 38 may be a heartbeat rate sensor, breath rate sensor, hygrometer, microphone, a weight sensor, and the like. The processor compares the metered values from the sensors, and compares each value to a predefined threshold. When a metered value passes beyond the threshold, an alert signal is transmitted to remote control 50 via wireless communication channel 60.

As per sensors such as heartbeat rate sensor, breath rate sensor, hygrometer, weight sensor, and the like, which meters a parameter such as heartbeat rate, the metered parameter is compared to a predefined threshold thereof, and if the metered value exceeds the threshold, an alert is sent to the remote control 50.

As per the microphone, the processor may use a DSP (Digital Signal Processing) processor (not shown) for detecting from the sampled audio signal if the infant is crying, the cry intensity, and the like.

According to another embodiment of the invention, the strength of the sampled sound is metered. If the cry's strength passes beyond a predefined threshold, then an alert signal is transmitted to the remote control 50.

In addition, the processor may transmit to the remote control a reminder for feeding the infant, changing the infant's diaper, and the like.

The multimedia system 42 comprises at least audio display circuitry for playing audio signals, and speakers, which preferably are disposed at the sides of the infant's head. The multimedia system may also use a display, such as screen 12, thereby allowing displaying movies. The multimedia system may use external memory in which are stored multimedia files, thereby allowing a parent to change the displayed content.

It should be noted that the processor does not necessarily have to be installed on the infant's belt, but in any place at the crib's side.

The remote control 50 comprises a processor 58, which gets input from operational buttons and displays output signals by output device(s) 52, such as a display and speaker. The operational buttons are used for setting the thresholds, reminders, and the like.

According to one embodiment of the invention, the remote control 50 is embodied by a smart phone running a corresponding software application. This spares the development time from manufacturers, using an additional device, and so on.

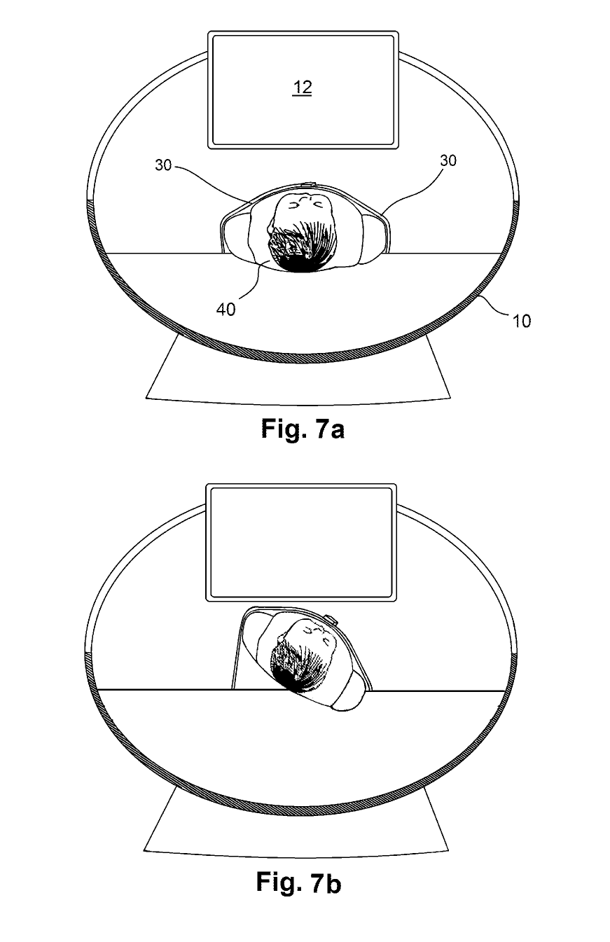

FIG. 7a is a latitudinal cross-section of an anti-SIDS infant crib in which the infant lies on his back, according to one embodiment of the invention.

FIG. 7b is a latitudinal cross-section of an anti-SIDS infant crib in which the infant tries to turn upside-down while the elastic belt prevents it, according to the same embodiment of the invention.

One of the major features of the invention is the ability to prevent an infant lying in the crib from rolling on his side or stomach. According to this embodiment, such means is an adjustable and elastic belt 30 connected to the bottom of the crib, on which the infant lies on. Keeping the infant lying on his back decreases the chance of the infant dying from SIDS.

As illustrated in FIG. 7b, the belt is tensed as being elastic, thereby preventing the infant from turning upside-down on his stomach. On the one hand, the infant can change its position, but on the other hand he cannot turn upside-down. One advantage of this embodiment is that the infant's hands cannot tangle with the belt. Of course, the elasticity of the belt should be adjusted to allow limited rolling, but not turning over by applying a power of an infant.

According to one embodiment of the invention, the results of the sensing are sent to a remote database. These results may be accumulated from a plurality of infants, and allow performing a research for deducing patterns and combinations of metered sensing that indicate an emergency satiation, especially SIDS. Then, these patterns can be sent to the processor of the crib, and be used for detecting situations in which the parents have to be alerted.

In the figures and/or description herein, the following reference numerals (Reference Signs List) have been mentioned:
numeral 10 denotes a crib;
numeral 12 denotes a screen;
numeral 14 denotes a mattress;
numeral 16 denotes a camera;
numeral 18 denotes a background light lamp;
numeral 20 denotes a processor, i.e., a circuitry that responds to and processes basic computing/electronic instructions;
numeral 22 denotes a baby bottle heater facility;
numeral 24 denotes a base of crib 10;
numeral 26 denotes a length counter;
numeral 28 denotes a track;
numeral 30 denotes an adjustable belt;
numeral 32 denotes a handle;
numeral 34 denotes a display on belt 30;
numeral 36 denotes a baby bottle;
numeral 38 denotes a sensor;
numeral 40 denotes an infant;
numeral 42 denotes multimedia system;
numeral 50 denotes a remote control;
numeral 52 denotes output device(s) of remote control 50, such as screen and speakers;
numeral 54 denotes a wrist belt of remote control 50;
numeral 56 denotes operational buttons;
numeral 58 denotes a processor;
numeral 60 denotes wireless communication;
numeral 62 denotes a microphone; and
numeral 64 denotes a Velcro fastener.

In the description herein, the following references have been mentioned: U.S. Pat. Nos. 8,333,560, 5,389,037, and 5,483,711.

The foregoing description and illustrations of the embodiments of the invention has been presented for the purposes of illustration. It is not intended to be exhaustive or to limit the invention to the above description in any form.

Any term that has been defined above and used in the claims, should to be interpreted according to this definition.

The reference numbers in the claims are not a part of the claims, but rather used for facilitating the reading thereof. These reference numbers should not be interpreted as limiting the claims in any form.

1. An anti-SIDS infant crib, comprising:
a crib (10);
a processor (20);
one or more SIDS-related sensors (38), installed at the crib's side, and being in communication with said processor (20);
an elastic and adjustable belt (30) surrounding an infant in said crib, for preventing said infant from turning upside-down while allowing a limited ability to said infant to turn over;
wherein said belt hosting one or more sensors that come in contact with an infant body or leotard;
wherein said processor being adapted to identify an SIDS alerting state by a combination and threshold of said sensors (38);
a remote control (50), in wireless communication (60) with said processor (20), said remote control being adapted for monitoring an operation of said processor (20), and alerting a user upon identifying an alerting state by said processor;
thereby providing to a parent SIDS-control and alerts of an infant lying in said crib.

2. An anti-SIDS infant crib according to claim 1, wherein said sensors (38) include (a) a camera (16) and microphone (62), for taking images of an infant lying in said crib, and displaying thereof on a remote display, and (b) warnings.

3. An anti-SIDS infant crib according to claim 1, further comprising a multimedia system (42), disposed at said crib's side, for displaying content to said infant.

4. An anti-SIDS infant crib according to claim 3, further comprising a screen (12), for displaying to said infant video presentations.

5. An anti-SIDS infant crib according to claim 4, further comprising an adjustable mattress (14), for allowing adapting thereof so that an infant lying in said crib can view said screen.

6. An anti-SIDS infant crib according to claim 4, further comprising a baby bottle heater facility (22) controllable by said processor (20), for heating a baby bottle (36) disposed therein.

7. An anti-SIDS infant crib according to claim 1, wherein said one or more sensors (38) that come in contact with an infant body or leotard include a heartbeat rate sensor.

8. An anti-SIDS infant crib according to claim 1, wherein said one or more sensors (38) that come in contact with an infant body or leotard include a breath rate sensor.

9. An anti-SIDS infant crib according to claim 1, wherein said one or more sensors (38) that come in contact with an infant body or leotard include an oxygen amount sensor in blood.

10. An anti-SIDS infant crib according to claim 1, wherein said one or more sensors (38) that come in contact with an infant body or leotard include a hygrometer, for detecting if a diaper of said infant needs to be changed.

11. An anti-SIDS infant crib according to claim 1, wherein said one or more sensors (38) that come in contact with an infant body or leotard include a thermometer, for metering a temperature of said infant.

12. An anti-SIDS infant crib according to claim 1, wherein said one or more sensors (38) include a thermometer, for metering a room temperature.

13. An anti-SIDS infant crib according to claim 1, wherein said one or more sensors (38) include a microphone, and said processor is further adapted to detect an infant's cry.

14. An anti-SIDS infant crib according to claim 1, wherein said one or more sensors (38) include a length meter in a form of a track (28) disposed at the inner side of the crib's walls, and a length counter (26) slideable along said track, thereby allowing measuring a height of said infant when lying in said crib.

15. An anti-SIDS infant crib according to claim 1, wherein said one or more sensors (38) include a weight meter, for metering a weight of said infant.

16. An anti-SIDS infant crib according to claim 1, further comprising means for storing results of measurements, thereby allowing diagnosing abnormal development of an infant lying in said crib.

17. An anti-SIDS infant crib according to claim 1, further comprising means for transmitting measurements' results to a remote database, for deducing combinations and thresholds in which an alert has to be issued, and updating said processor of thresholds and combinations of alerting states.

18. An anti-SIDS infant crib according to claim 1, wherein said belt further comprising a vibration mechanism for vibrating said belt upon identifying said SIDS alerting state.

19. An anti-SIDS infant crib according to claim 1, wherein said belt further comprising a display, for displaying measurements, states, and instructions.
